(19) United States
(12) Patent Application Publication
(10) Pub. No.: US 2026/0014307 A1
(43) Pub. Date: Jan. 15, 2026

(54) SHUNT FOR USE IN DIALYSIS

(71) Applicant: Stanley Batiste, Granite Bay, CA (US)

(72) Inventor: Stanley Batiste, Granite Bay, CA (US)

(21) Appl. No.: 18/955,738

(22) Filed: Nov. 21, 2024

Related U.S. Application Data
(63) Continuation-in-part of application No. 18/202,010, filed on May 25, 2023.
Application No. 18/202,010 is a division of application No. 16/659,386, filed on Oct. 21, 2019, now Pat. No. 12,042,592.
(60) Provisional application No. 62/748,280, filed on Oct. 19, 2018.
(60) Provisional application No. 63/602,297, filed on Nov. 22, 2023.

(51) Int. Cl. A61M 1/36

(52) U.S. Cl. CPC A61M 1/3655; A61M 1/3661; A61M 2205/0216; A61M 2205/3334; A61M 2206/10

(57) ABSTRACT

A shunt is provided for use in dialysis. The shunt includes a flexible tube configured to connect an artery to a vein. The flexible tube includes a first end configured to be connected to the artery of the patient, a second end configured to be connected to the vein of the patient, and a wall extending from the first end to the second end to form the tube. The wall has an inner surface, an outer surface, and a distance between the inner and outer surfaces. The distance varies between a first distance at both the first end and the second end, and a larger second distance at a region between the first end and the second end, the larger second distance forming a stenosis in the tube which due the second distance being larger than the first distance resists kinking at the stenosis.

### BACKGROUND OF THE INVENTION

### 1. Field of the Invention

The invention relates generally to dialysis grafts and in particular to an adjustable graft using aspiration.

### 2. Related Art

There are currently more than 600,000 patients in the United States with end-stage renal disease (ESRD) and many times more than that throughout the world. ESRD accounts for approximately 6.4% of the overall Medicare budget at over $23 billion dollars in the US in 2006. Patients with end stage renal disease have lost their normal kidney function and as a result require dialysis to substitute the function of the kidney cleansing the blood. There are two types of dialysis: hemodialysis and peritoneal dialysis. For the purposes of this overview, we will primarily be focused on hemodialysis and later discuss briefly the topic of peritoneal dialysis.

Hemodialysis requires that large volume blood access and exchange be consistently available to sustain the life of the patient. Typically, a dialysis patient will require 3-4 hours of dialysis three days a week. The challenge with providing hemodialysis is maintaining access to large volumes of blood when a body constantly fights attempts to keep access available by healing closed such access. Currently there are three ways to provide hemodialysis: dialysis catheters, arterial venous fistulas (AVFs) and arterial venous grafts (AVGs). Although used worldwide, catheters are known not to be efficient for long term dialysis. Unfortunately, catheters have very short patency rates and high rates of infection. For these reasons dialysis guidelines strongly oppose catheter use, other than for short term use, until fistula or graft placement is available.

AVGs and AVFs are synthetic and natural conduits respectively that are surgically placed to provide long term dialysis access. Both provide large diameter targets that can be easily accessed with large needles for blood exchange. These conduits are commonly placed in the arm with the furthest point attached to the patient's artery and then are directly attached to the vein for blood flow return. The high arterial blood pressure and flow is shunted directly to the vein providing dilatation of the vein or graft and large volume blood flow. Although these methods provide excellent means of access, both have limitations with regard to sustaining long term patency. The patency rates are much greater than that of a catheter however overall are relatively poor when considering the few years gained in a patient's life. It has been noted that there is only 50% shunt patency at one year and less than 25% at 2 years. Not only does this create a huge burden on the cost of healthcare but more importantly, once access is no longer available, a new access point must be created to sustain a patient's life.

A thorough description of the reason for dialysis fistula and graft failure is beyond the scope of this document. The fundamental problem is that the flow dynamics created by these artificial conduits are not normal to our bodies. The change is detected by the body and the normal physiologic defenses become involved and attempt to return the system to normal leading to graft or fistula failure. Failure of the graft generally means that the graft or fistula ceases to maintain flow. Once occluded the graft becomes full of blood which is static which subsequently becomes thrombus. Once failure occurs, the patient loses the ability to have hemodialysis until access function is restored.

From the discussion that follows, it will become apparent that the present invention addresses the deficiencies associated with the prior art while providing numerous additional advantages and benefits not contemplated or possible with prior art constructions.

### SUMMARY OF THE INVENTION

A shunt is provided for use in dialysis. The shunt includes a flexible tube configured to connect an artery of a patient to a vein of a patient. The flexible tube includes a first end configured to be connected to the artery of the patient, a second end configured to be connected to the vein of the patient, and a wall extending from the first end to the second end to form the tube. The wall has an inner surface, an outer surface, and a distance between the inner and outer surfaces. The distance varies between a first distance at both the first end and the second end, and a larger second distance at a region between the first end and the second end, the larger second distance forming a stenosis in the tube, which due the second distance being larger than the first distance resists curving at the stenosis.

The distance within the region may vary from ends of the region to a location between the ends. The region may have a pocket for including a different material than a material of the flexible tube. The flexible tube may also have a concave portion on an outer surface proximate the region.

Further objects, features, and advantages of the present invention over the prior art will become apparent from the detailed description of the drawings which follows, when considered with the attached figures.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 is an illustration of a short flow restrictor and long flow restrictor.

FIG. 2A, FIG. 2B, and FIG. 2C illustrate a linear section of an AV graft (tube) and grafts with flow restrictors applied.

FIG. 3 illustrates a short flow restrictor on a stent expanded and collapsed.

FIG. 4 illustrates a long flow restrictor on a stent expanded and collapsed.

FIG. 5A and FIG. 5B illustrate a stent deployment catheter with collapsed stent and deployed stent.

FIG. 6 illustrates a collapsed (unexpanded) and expanded balloons.

FIG. 7A is an illustration of a collapsed balloon advancing into graft with a short restrictor.

FIG. 7B is an illustration of a balloon positioned within the short restrictor.

FIG. 7C is an illustration of a balloon inflated expanding restrictor.

FIG. 7D is an illustration of a balloon deflated.

FIG. 7E is an illustration of a balloon removed and an expanded flow restrictor.

FIG. 8A is an illustration of a deflated balloon advancing into graft with a long flow restrictor.

FIG. 8B is an illustration of an inflated balloon expanding the long flow restrictor.

FIG. 8C is an illustration of a deflated balloon removal and the expanded restrictor 20.

FIG. 9A is an illustration of a deflated balloon within a flow restrictor.

FIG. 9B is an illustration of an expanded balloon and a fractured-expanded flow restrictor 10a.

FIG. 9C is an illustration of a balloon deflated and an expanded-fractured restrictor.

FIG. 9D is an illustration of a graft with an expanded-fractured restrictor.

FIG. 10A is an illustration of a deflated balloon advancing into the flow restrictor 10b.

FIG. 10B is an illustration of a deflated balloon within a flow restrictor.

FIG. 10C is an illustration of a balloon expanded within a flow restrictor.

FIG. 10D is an illustration of a balloon deflated and an elastic restrictor.

FIG. 10E is an illustration of a balloon removal and an elastic restrictor regaining shape.

FIG. 11 illustrates one such embodiment that includes a stent tube having multiple bands of various diameters.

FIG. 12 illustrates an embodiment with a mirror set of bands.

FIG. 13 illustrates an embodiment with bands having a greater width.

FIG. 14 as shown is a dialysis machine with inflow and outflow lines and graft with proximal flow limb and distal flow limb having a mid-graft stenosis.

FIG. 15 illustrates an embodiment of the invention having the mid-graft stenosis within the apex of the graft between the arterial limb and the venous end.

FIG. 16 illustrates one embodiment where the mid-graft stenosis is constructed of thickened material with smooth walled tapering.

FIG. 17 illustrates an embodiment where the mid-graft stenosis has a smooth inner diameter taper and an exterior taper design with thickened wall for support.

FIG. 18 illustrates an embodiment shown with a mid-graft smooth taper with hollow inner wall to allow balloon expansion.

FIG. 19 as illustrated is an embodiment with a smooth taper mid-graft stenosis and a material within the wall which is made of a different material than the material which forms the wall.

FIG. 20 as illustrated is an embodiment with a mid-graft smooth taper where the thickened wall is composed of hollowed out cells or filled with material.

### DETAILED DESCRIPTION OF THE PREFERRED EMBODIMENTS

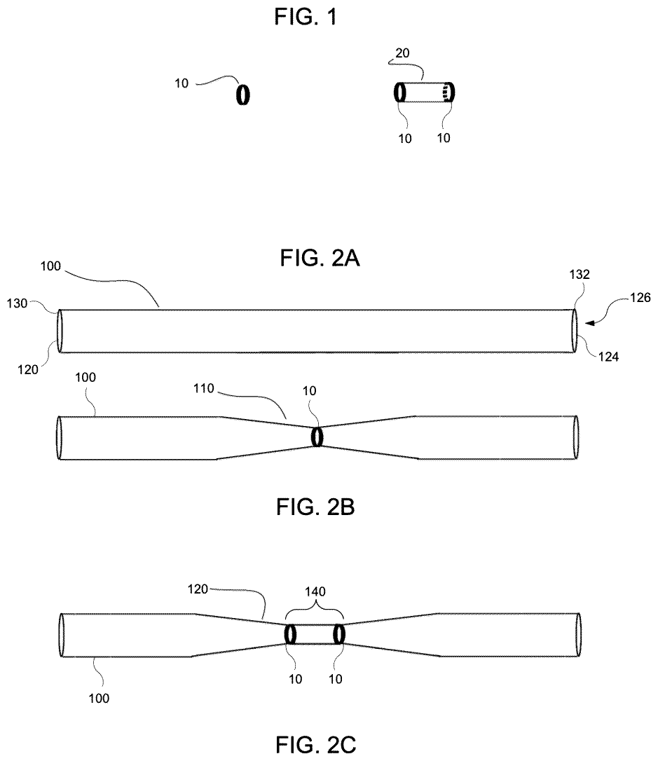

In one or more embodiments the invention utilizes a band to create external pressure on elements in order to narrow the inner lumen of a dialysis shunt thereby creating a stenosis that acts as a flow restrictor. The restricted flow then creates the perfect hemodynamic conditions within the shunt helping to eliminate shunt failure and improve flow to the extremities while decreasing cardiac output. As shown in FIG. 1 there are two sizes of bands or restrictor length represented; short (10) and long (20). Bands of different sizes may be used, such as wider versions shown in FIG. 13. It is also contemplated that that the long restrictor length may be formed by two bands 10, one at each end of the narrowed restrictor area, or multiple bands spaced for form the narrowing. The restrictor bands can be composed of materials that have the properties of expandable, fracturable and elastic. In one embodiment the restrictor is able to be expanded in diameter and also be reduced in diameter.

FIG. 2A illustrates an example tube 100 that may be used to form the shunt. The term shunt and graft may be used interchangeably herein. The tube 100 has an outer wall and an inner opening 126 that extends through the tube. The tube has an inlet end 130 (first end) that receives blood flow and an outlet end 132 (second end) that is for an outflow of blood. The tube 100 may be of different diameters to match the diameter of a vessel to which the tube will connect. It is contemplated that in one embodiment the inlet end is configured to connect to an artery while the outlet end is configured to connect a vein. The tube 100 may be any length and may be trimmed to cut to a suitable length. It is also contemplated that bands 10 may be manufactured with bands on the tube 100 or the bands may be placed on the tube at the desired location just prior to or during a medical procedure.

FIG. 2B illustrates the restrictor band 10 deployed on the outside of the hollow member (tube 100) of the arterial venous graft that is surgically placed within a patient. Once placed in a patient, blood flow through the graft will be limited due to the restriction 110 thereby improving conditions while maintaining the ability to have dialysis. The conditions that are improved include, but are not limited to, a reduction or elimination of normal vein stenosis at the output of the graft and stealing of blood from the artery.

FIG. 2C illustrates an embodiment with two bands 10 on the tube 100 to create a longer restricted region 140. One or more additional bands may be placed in the restricted region 140 to maintain the narrowing of the restricted region.

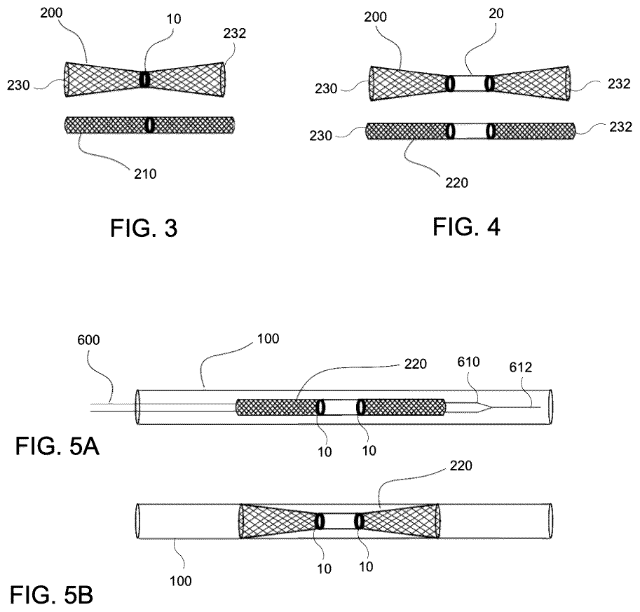

FIG. 3 illustrates an endovascular stent with a restrictor band. The stent 200 is shown in a contracted and expanded position. As shown, the stent 200 is provided with a restrictor band 10. The stent 200 has a first end 230 and a second end 232 and is configured to be placed inside a blood vessel of a patient. The short flow restrictor 10 on a stent expanded and collapsed 210 is shown. Endovascular stents are understood by those of ordinary skill in the art and as such they are not described in detail herein. The restrictor band in the stent 200 serves the same purpose as when used in connection with a shunt.

FIG. 4 illustrates the restricted section 20 placed on the outside portion of an endovascular stent 200 both within the collapsed position and the deployed expanded position. As with FIG. 3, the method of placement is similar to traditional stent placement where percutaneous access is first gained, followed by wire access, followed by collapsed stent advancement to the desired location in the vessel. Once positioned, the stent 200 is deployed. With the restrictor bands 10 in place, the stents outer portions (ends 230, 232) will open to oppose the vessel wall. The restrictor located in the middle, or between the ends, or away from the ends, will create radial pressure within the central portion of the stent prohibiting its central expansion, thereby creating a restriction. This restrictor will maintain a narrowing, creating the desired hemodynamic effects.

FIG. 5A illustrates a stent deployment catheter 600 with collapsed stent 220 and deployed stent. This is but one possible system for deployment. The catheter 600 is placed inside the vessel and advances to the deployment location. The stent 220 includes the bands 10 which form the restriction. The catheter is inserted into a vessel or a shunt tube 100 and the stent 220 positioned to a location at which the stent will be placed. Element 100 may be a shunt tube or a vessel of a patient. In this embodiment, the stent 220 is secured to a balloon 610 which is guided by a wire 612. When the balloon is expanded, as shown in FIG. 5B, the stent 220 is secured to the wall of the vessel to maintain itself in place. The outer ends of the stent 220 expand while the restricted portion between the bands 10 does not expand and thus forms the restricted section. The center area of the balloon 610 may not expand thus leaving the narrow area between the bands un-expanded.

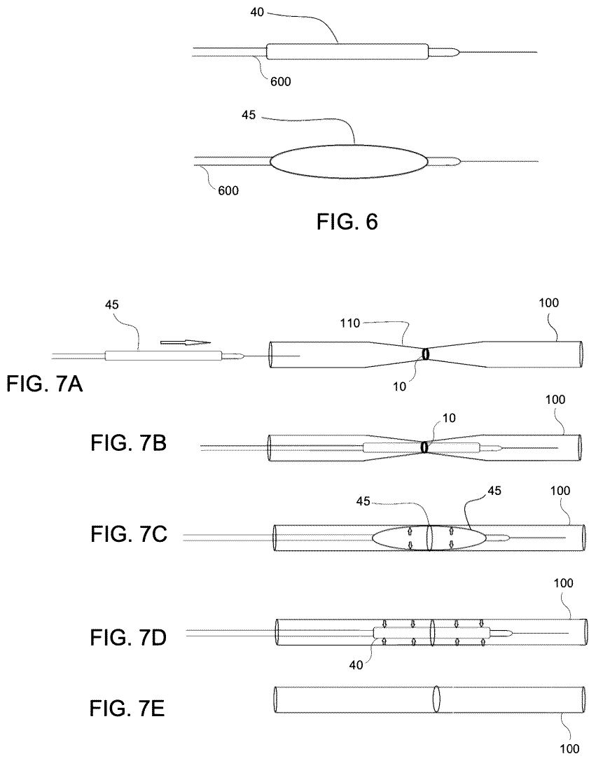

FIG. 6 illustrates an unexpanded balloon 40 in connection with a catheter 600 and an expanded balloon 45 in connection with a catheter 600. Catheter based balloons are understood by those of ordinary skill in the art and as such are not described in detail herein.

Once created or deployed various clinical situations may dictate modification of the restrictor whether permanent or temporary. The modification may include the diameter of the restriction or the length of the restricted section. Thus, the design anticipates the need to alter the restrictor once placed. The restrictor, bands 10 may be altered with various materials depending on the clinical situation and hence the restrictors bands may be expandable, fracturable or elastic. In some embodiments, the restrictor bands 10 return, or can be manipulated to return, to a narrower diameter after being expanded.

As shown in FIG. 7A through FIG. 7E, the expandable restrictor is described where the non-expanded restrictor band 10 is first used within the shunt FIG. 7A to create a restricted section 110 in the shunt tube 100. When clinically needed a balloon 40 can be passed using endovascular technique into the center section of the restrictor band 10. Once the balloon 40 is inflated to become an inflated balloon 45, the restrictor band 10 is stretched as shown in FIG. 7C. When the balloon is deflated as shown in FIG. 7D and removed, the restrictor band 10 maintains its expanded shape as shown in FIG. 7E. This system allows a narrowing to be established in the tube through which blood flows and then if needed, remove the restriction to restore unrestricted blood flow. All this may occur with minimally invasive catheter and balloon techniques.

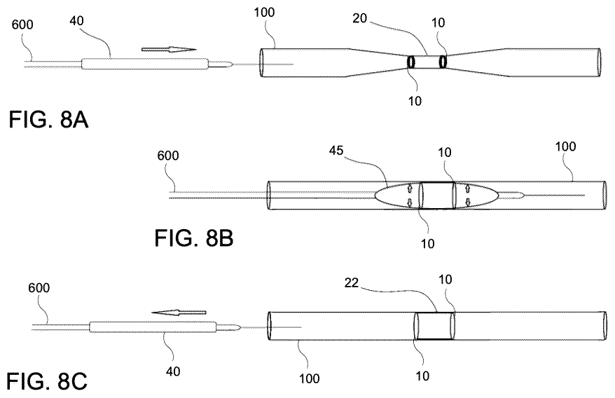

FIG. 8A, FIG. 8B, and FIG. 8C illustrate a system and process as shown in FIG. 7A through FIG. 7E but with an expanded restricted area formed by two restrictor bands 10. As compared to FIG. 7A through FIG. 7E, similar elements are identified with identical reference numbers. In this embodiment, the expanded balloon 45 may have a wider area to expand both bands 10. This is similarly shown when the restrictor was initially deployed using the longer restrictor FIG. 8A through FIG. 8C.

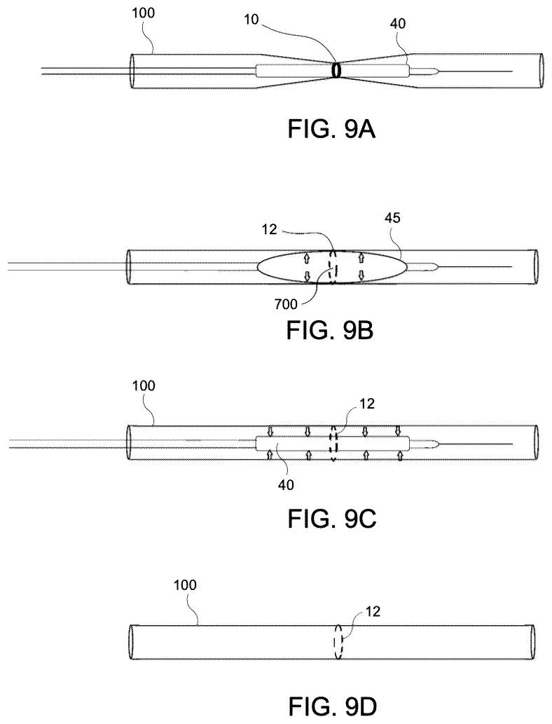

In a second embodiment, shown in FIG. 9A through FIG. 9D, a fracturable restrictor band 10 is used again creating a narrowing within the shunt. When a balloon is passed and inflated within the fracturable restrictor, the ring fractures at fracture points 700 as shown in FIG. 9B. Preset fracture points 700 that break or fracture to allow the release of pressure and associated radial force existing tube, and previously restrained by the fracturable restrictor band 10. Once fractured, the band is referred to as a fractured band 12. As shown in FIG. 9C, the balloon 40 is deflated for removal and the fractured band 12 no longer creates a stenosis. Then, in FIG. 9D, the shunt tube 100 is unrestricted. This allows expansion of the shunt tube 100 eliminating the narrow area and blood flow resistance. This creates a permanent loss of narrowing.

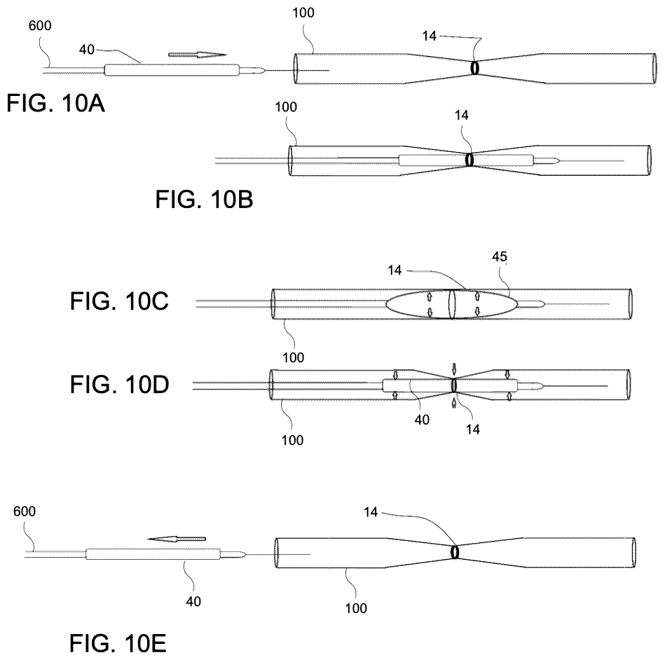

A third embodiment of the material design is that of a resistor band 14 made of elastic materials. As shown in FIG. 10A through FIG. 10E, the restrictor band 14 is created or deployed and once the need arises to alter the restrictor band 14 it is temporarily opened. As shown in FIG. 10C, the balloon 45 is inflated to expand the restrictor band 14. This expands the stenosis and based on the elasticity and properties of the restrictor band 14 the band may stay at a larger diameter for different periods of time. Once the balloon 40 is deflated, the elastic material of the restrictor band 14 reform to its original state again acting as a flow restrictor. The return to the narrow shape may occur rapidly or after a period of time. This may be useful when balloon angioplasty is needed in other parts of the shunt to help maintain patency, but the stenosis is still desired after the angioplasty. The elastic restrictor band 14 may be made of any material that is expandable and then returns to its narrowed diameter shape rapidly or slowly after expansion.

In one embodiment, the shunt is equipped with two or more fracturable restrictor bands such that one or more of the restrictor bands have a different diameter. In a basic embodiment, the first restrictor band has an inner diameter that is less than the inner diameter of the second restrictor band. Both are located around the outer surface of the shunt. In use, the shunt is paced in a patient which establishes a restriction or stenosis in the shunt thereby reducing blood flow. If, over time, it is determined that the restriction is too great, then a balloon may be directed through a blood vessel to the shunt and filled with gas or liquid to increase the diameter of the balloon. The diameter of the balloon may be increased sufficiently to fracture the first restrictor band, while leaving the second restrictor band unaffected. Thus, after fracturing the first restrictor band, the inner diameter of the opening is increased to the diameter of the second restrictor band. If the doctor again determines the blood flow restriction is too great, then a balloon may be inserted inside the second restrictor to fracture the second structure thereby again increasing the inner diameter of the shunt. This may occur with more than two restrictor bands to create additional degrees of control over the restriction size.

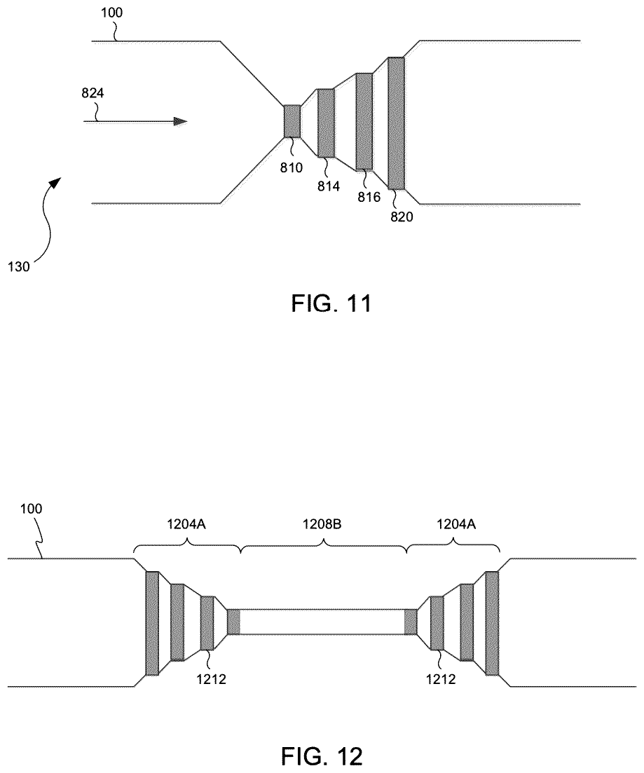

FIG. 11 illustrates one such embodiment that includes a stent tube having multiple bands of various diameters. This is but one possible configuration and as such other embodiments and configurations are possible, that which do not depart from the claims that follow. In this embodiment the shunt tube 100 includes an intake end configured to accept blood flow 824 as shown by the arrow. Any number of bands are possible but in this embodiment four bands 810, 814, 816, 820 are shown. A first band 810 has the smallest diameter, referred to herein as a first diameter. A second band 814 has a diameter greater than the first band 810, referred to herein as a second diameter. A third band 816 has a diameter greater than the second band 814, referred to herein as a third diameter. A fourth band 820 has a diameter greater than the third band 816, referred to herein as a fourth diameter.

In operation, a balloon (not shown) or other expansion device, may be inserted into the tube 100 and expanded sufficiently to fracture or otherwise expand the first band 810 thereby changing the diameter of the stenosis from the first diameter to the second diameter. If that stenosis is too small, then the process can be repeated at a later time to break the second band 814. This process may repeat to provide adjustability in the amount of narrowing in the stenosis including no narrowing by breaking all bands.

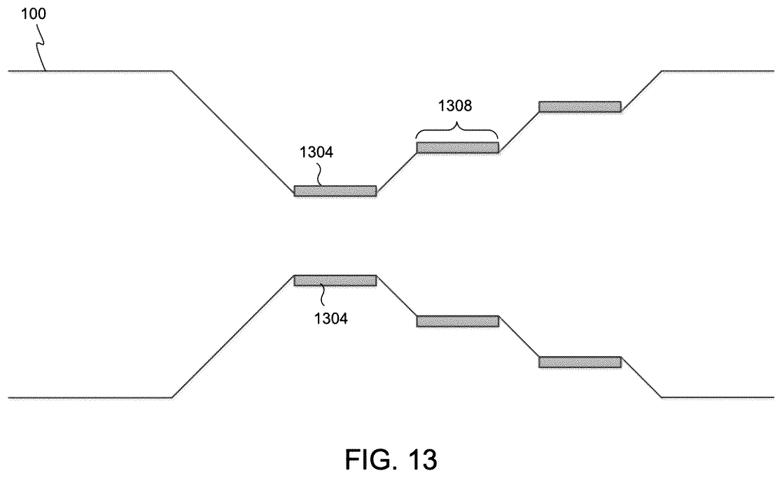

FIG. 12 illustrates an embodiment with a mirror set of bands. The first set of bands 1204A is duplicated with a second set of bands 1204B. The bands may be separated by a separation distance 1208. Operation of this embodiment is as described above. The number of bands may vary from embodiment to embodiment. The value of the separation distance 1208 may vary to control blood flow dynamics as described herein. As the bands 1212 are broken, expanded, stretched, or separated, the tube will expand outward to a diameter determined by the band of the smallest diameter.

FIG. 13 illustrates an embodiment with bands having a greater width. It is also contemplated that the length of the stenosis may be determined by the width of the band. As such, the bands may be of different thickness or width. As shown in FIG. 13, the bands 1304 shown in cross section, are wider than those shown in the other embodiments. Varying the stenosis length 1308 will adjust blood flow dynamics. As with the other embodiments shown and described herein, the bands may be expanded to adjust the diameter of the stenosis. It is also contemplated that the width of each band may vary even for a single shunt tube 100. Thus, one band may be narrow, the next band wide, and the next band wider. Thus, each band would establish a different stenosis length.

It has been shown that surgical improvements to failing dialysis grafts can improve long term patency and as a result increase the lifespan of patients. One successful surgical procedure involves creating a narrowing within the mid aspect of a dialysis graft or fistula. This procedure is referred to as precision banding and requires that the graft is surgically exposed, and a suture is then applied around the graft and tightened, thus narrowing the lumen and creating a stenosis. This stenosis decreases pressure, flow and pulsation improving the hemodynamic properties of the fistula or graft.

The disclosed concept utilizes thickened and shaped materials to create properties of the stenosis that cannot be achieved with conventional thin-walled materials. The proposed thickened and shaped materials improve the ability to restore function in the grafts if failure occurs. Grafts typically have a short-term patency rate, and although the rate may improve with the stenosis in the modified graft, failure within 2 years is common. Once a graft fails, pharmacological (lytic medication) and/or mechanical therapy will be needed to restore flow as needed with dialysis.

When mechanical therapy is employed, it typically uses balloon angioplasty to open up areas of narrowing that many times are the reason for graft failure. These narrowings can occur within the graft itself and/or in the patient's native veins. The disclosed concept proposes a controlled narrowing to solve drawbacks in the art, such that a single specific location may be targeted for improved dialysis. In contrast, the pathologic narrowings that develop within standard grafts decrease dialysis function and may cause graft failure. Therapy may require performing angioplasty within the disclosed concept.

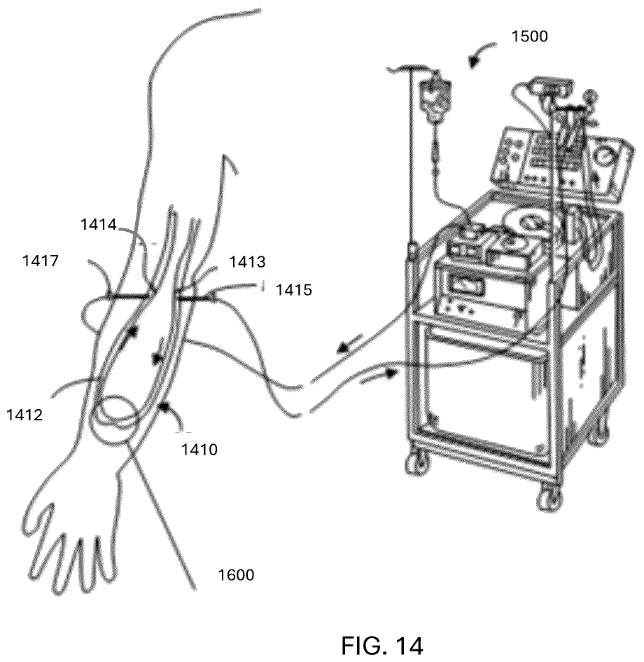

Additionally, the disclosed grafts, as will be discussed below in connection with FIG. 14 through FIG. 20, therefore, are preferably able to withstand expansion via angioplasty balloon and subsequently reform to their initial configuration afterwards. For this subsequent reformation to occur in the graft after balloon angioplasty expansion therapy, the grafts have advantageous configurations and expanded wall thicknesses, as the standard ePTFE materials currently only utilize uniform thin-walled narrowing.

A second limitation of current AV Graft narrowing design is that of surgical graft placement and kinking. Uniform thin-walled design, when placed in the standard U-shape, tends to kink at the point of narrowing. The disclosed concept provides added stiffness and body to provide for a more rigid design which eliminates kinking.

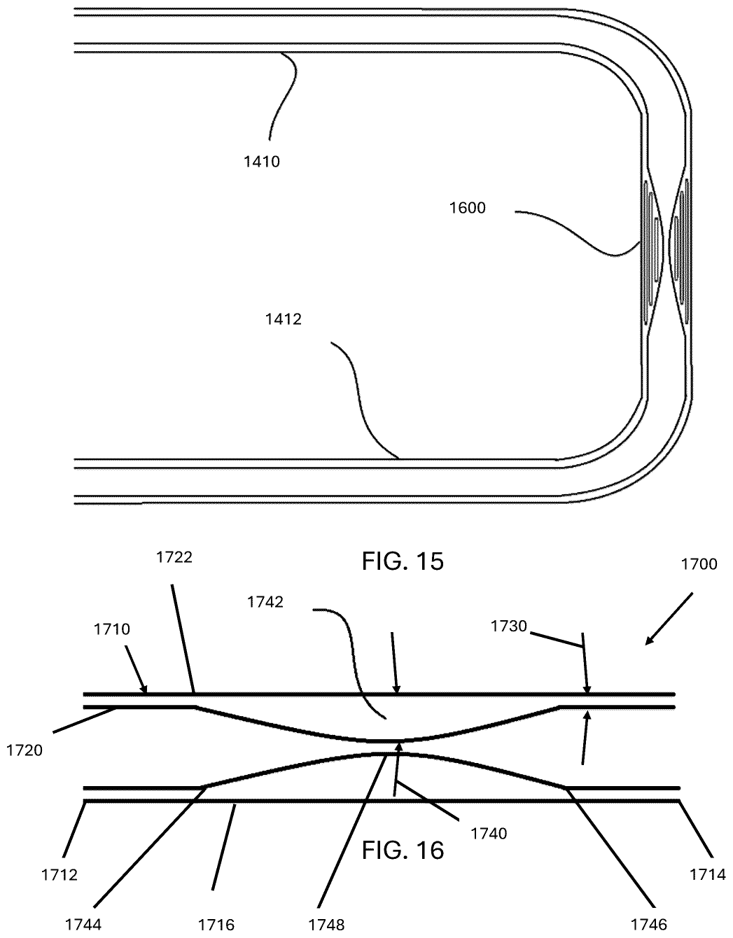

FIG. 14 illustrates an attachment of a dialyzer unit 1500 with AV graft access points 1415 and 1417, and a mid-graft stenosis 1600 (also shown in FIG. 15), in accordance with one non-limiting embodiment of the disclosed concept. High pressure from the access point 1417 within the proximal graft 1412 benefits flow to the dialyzer 1500, and low pressure distal to the mid-graft stenosis 1600 in the outflow limb 1410 helps low resistance flow from the dialyzer to the patient.

In one example, as shown in FIG. 15, a premanufactured stenosis 1600 between the proximal and distal ends of an arterial venous dialysis graft is designed to create resistance to flow optimizing dialysis and preventing high-flow related pathologies. The stenosis 1600 is shown isolated in FIG. 15 positioned between an inflow limb 1412 and an outflow limb 1410. High pressure flow coming into inflow limb 1412 meets the resistance of the stenosis 1600 and the outflow is dampened with decreased flow rate and pressure in the outflow limb 1410.

One example embodiment of the disclosed concept is depicted in FIG. 16, which shows an example shunt (e.g., arterial venous shunt 1700) for use in dialysis with the dialyzer unit of FIG. 14. The shunt 1700 may be a unitary component made from a single piece of material (i.e., a molded or formed piece). The shunt 1700 preferably includes a flexible tube 1710 configured to connect an artery of a patient to a vein of the patient. The flexible tube 1710 includes a first end 1712 configured to be connected to the artery of the patient, a second end 1714 configured to be connected to the vein of the patient, and a wall 1716 extending from the first end 1712 to the second end 1714 to form the tube 1710.

The wall 1716 of the flexible tube 1710 of FIG. 16 preferably has an inner surface 1720, an outer surface 1722, and a distance between the inner and outer surfaces, the distance varying between a first distance 1730 at both the first end 1712 and the second end 1714, and a larger second distance 1740 at a region 1742 between the first end 1712 and the second end 1714. In one example, the larger second distance 1740 forms a stenosis in the tube 1710 which due the second distance 1740 being larger than the first distance 1730 resists kinking at the stenosis.

In one example, the region 1742 of the tube 1710 has a first end 1744, a second end 1746 opposite the first end 1744 of the region 1742, and a location 1748 between the first and second ends 1744 and 1746. As shown in FIG. 16, the second distance 1740 increases from the first end 1744 of the region 1742 to the location 1748, and also from the second end 1746 of the region 1742 to the location 1748. In other words, the entire region 1742 may generally define an hour-glass shaped opening in an interior of the wall 1716, with the location 1748 being located midway between the first and second ends 1744 and 1746. Accordingly, the stenosis if formed with smooth inner tapering and has a thickened wall in the region 1742 that can tolerate high pressure balloon angioplasty and recover its tapering without suffering damage as with thin-walled structures.

Continuing to refer to FIG. 16, it will be appreciated that the second distance 1740 is configured to be at least two times as large as the first distance 1730, at least at the location 1748, and also at other places extending from the location 1748 to proximate the first and second ends 1744 and 1746 of the region 1742. This increased distance, or width or thickness of the region 1742, advantageously allows for the aforementioned stenosis. It is also contemplated that the first distance 1730 may be between 0.9-1.1 millimeters, and the second distance 1740 may be at least 2.5 millimeters at the location 1748. Additionally, in one example, a ratio of an inner diameter of the inner surface 1720 at the first and second ends 1712 and 1714, to an inner diameter of the inner surface 1720 at the location 1748 (e.g., at the stenosis) is preferably between 1.25-3.5, and more preferably between 1.6-2.3.

Moreover, as shown in FIG. 16, the wall 1716 of the tube 1710 preferably has a constant outer diameter between the first and second ends 1712 and 1714 of the flexible tube 1710. It will be appreciated having the shunt 1700 formed with a constant outer diameter, in the example of FIG. 16, advantageously prevents a dead space from existing where fluid can build up during a dialysis procedure.

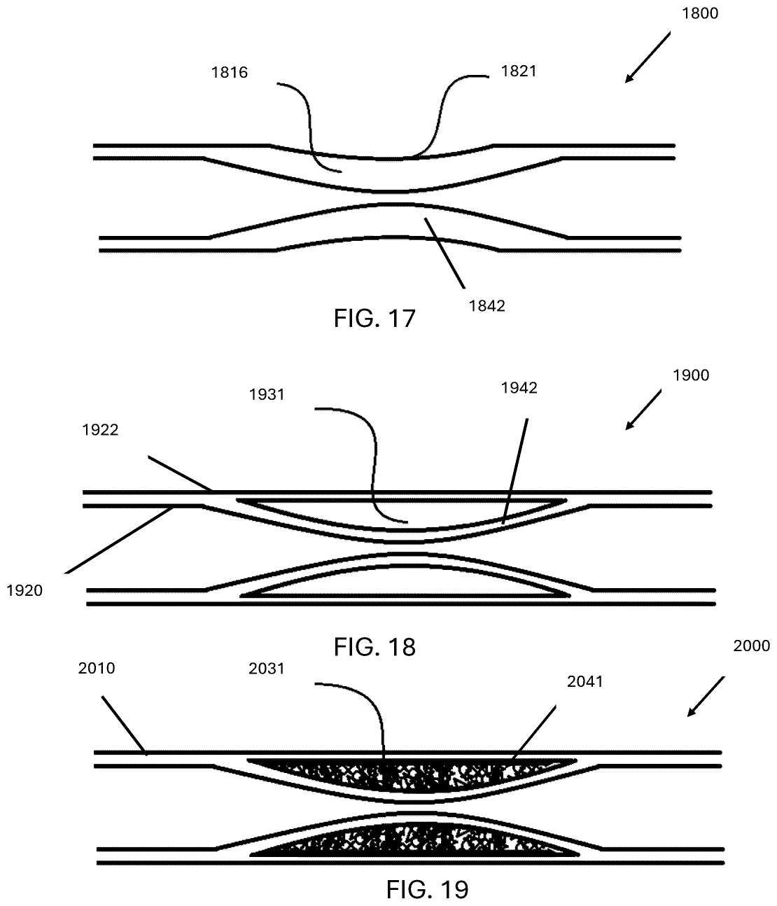

FIG. 17 through FIG. 20 depict other shunts (e.g., arterial venous shunts 1800 and 1900 and 2000 and 2100), each of which is configured similar to the shunt 1700 (FIG. 16), and wherein like numbers represent like features. For example, each of the shunts 1800 and 1900 and 2000 and 2100 preferably includes walls with varying distances between inner and outer surfaces, such that in regions between ends of the shunts 1800 and 1900 and 2000 and 2100, a stenosis is formed. The corresponding regions of the shunts 1800 and 1900 and 2000 and 2100 have profiles (e.g., defining hour-glass openings, etc.) the same as of the shunt 1700. As such, for economy of disclosure, additional features of the shunts 1800 and 1900 and 2000 and 2100 which are similar to those of the shunt 1700 will not be repeated below.

Regarding the shunt 1800, FIG. 17 shows an additional embodiment with the wall 1816 being thickened, and with the wall 1816 having a concave portion 1821 proximate the region 1842. Furthermore, the concave portion 1821 faces away from a central opening of the flexible tube. In this manner, expansion with balloon angioplasty stretches the outer lumen of the shunt 1800 to a lesser degree yet the volume of material prevents failure and retains its normal shape in neutral conditions post balloon therapy.

Regarding the shunt 1900, FIG. 18 illustrates another embodiment where the flexible tube further has a pocket 1931 between an outer surface 1922 of the wall and an inner surface 1920 of the wall, with the pocket 1931 being proximate the region 1942. As shown, the thickened inner material (e.g., the material from FIG. 16 and FIG. 17) has been replaced with a fluid or gas (e.g., compressible air). In other words, the pocket 1931 preferably has a gas located therein. In this manner, the embodiment depicted in FIG. 18 allows for balloon angioplasty if needed where the inner tapered stenosis can be expanded with the partial compressed hollowed region then reforming its shape after balloon therapy from the fluid or gas expansion.

FIG. 19 shows another shunt 2000 which has a pocket 2031 between inner and outer surfaces. As shown, the pocket 2031 has a material 2041 located therein, with the material 2041 preferably being different than a material that forms the flexible tube 2010. In one example, the material 2041 located in the pocket 2031 is more elastic than the material which forms the flexible tube 2010, and may be a rubber composite or other material that will deform during angioplasty dilatation treatment and then recover it neutral shape reforming the mid-graft taper after balloon deflation. In another example, the material 2041 located in the pocket 2031 has a first durometer value, and the material which forms the flexible tube 2010 has a second durometer value less than the first durometer value. As a result, the likelihood of kinking is desirably minimized.

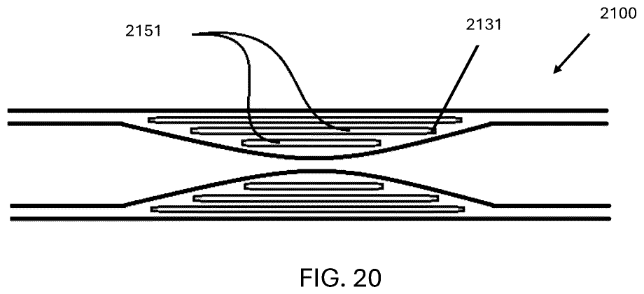

FIG. 20 shows the shunt 2100, which includes at least one pocket 2131 containing a number of cells 2151 located therein, with the cells 2151 containing at least one of a fluid, a gas, and a rubber material. In FIG. 20 the cells 2151 can be compressed during therapy and reform after balloon deflation.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A shunt for use in dialysis, the shunt comprising:
a flexible tube configured to connect an artery of a patient to a vein of the patient, the flexible tube comprising:
a first end configured to be connected to the artery of the patient;
a second end configured to be connected to the vein of the patient; and
a wall extending from the first end to the second end to form the tube, the wall having an inner surface, an outer surface, and a distance between the inner and outer surfaces, the distance varying between a first distance at both the first end and the second end, and a larger second distance at a region between the first end and the second end, the larger second distance forming a stenosis in the tube which due the second distance being larger than the first distance resists kinking at the stenosis.

2. The shunt of claim 1, wherein the second distance is at least two times as large as the first distance.

3. The shunt of claim 1, wherein the first distance is between 0.9-1.1 millimeters, and wherein the second distance is at least 2.5 millimeters at a location between first and second ends of the region.

4. The shunt of claim 1, wherein a ratio of an inner diameter of the inner surface at the first and second ends to an inner diameter of the inner surface at a location in the region is between 1.25-3.5.

5. The shunt of claim 4, wherein the ratio is between 1.6-2.3.

6. The shunt of claim 1, wherein the region has a first end, a second end opposite the first end of the region, and a location between the first and second ends of the region, and wherein the second distance increases from the first end of the region to the location, and from the second end of the region to the location.

7. The shunt of claim 6, wherein the location is midway between the first and second ends of the region.

8. The shunt of claim 6, wherein the wall has a constant outer diameter between the first and second ends of the flexible tube.

9. The shunt of claim 6, wherein the wall of the flexible tube has a concave portion proximate the region, the concave portion facing away from a central opening of the flexible tube.

10. The shunt of claim 1, wherein the flexible tube further has a pocket between an outer surface of the wall and an inner surface of the wall, the pocket being proximate the region.

11. The shunt of claim 10, wherein the pocket has a gas located therein.

12. The shunt of claim 10, wherein the pocket has a material located therein, the material being different than a material that forms the flexible tube.

13. The shunt of claim 12, wherein the material located in the pocket is more elastic than a material which forms the flexible tube.

14. The shunt of claim 12, wherein the material located in the pocket has a first durometer value, and wherein the material which forms the flexible tube has a second durometer value, the second durometer value being less than the first durometer value.

15. The shunt of claim 10, wherein the pocket contains a number of cells located therein, the cells containing at least one of a fluid, a gas, and a rubber material.

16. The shunt of claim 1, wherein the shunt is an arterial venous shunt.
